(19) United States
(12) Patent Application Publication
(10) Pub. No.: US 2014/0074254 A1
(43) Pub. Date: Mar. 13, 2014

(54) LATERALLY AND ROTATABLY ADJUSTABLE ALIGNMENT DEVICE WITH OPTIONAL SCALES AND SECURE CONNECTIONS

(71) Applicant: Michael J. Curtis, West De Pere, WI (US)

(72) Inventor: Michael J. Curtis, West De Pere, WI (US)

(21) Appl. No.: 13/842,650

(22) Filed: Mar. 15, 2013

Related U.S. Application Data
(60) Provisional application No. 61/698,389, filed on Sep. 7, 2012.
(60) Provisional application No. 61/710,441, filed on Oct. 5, 2012.
(60) Provisional application No. 61/766,339, filed on Feb. 19, 2013.

(51) Int. Cl. A61F 2/76

(52) U.S. Cl. CPC A61F 2/76
USPC 623/38

(57) ABSTRACT

The present invention relates to an offset alignment device with a longitudinal axis that is capable of singularly offsetting two prosthetic components a selected distance along an alignment axis that is selectably oriented in a plane that is generally perpendicular to the longitudinal axis, and is also capable of adjusting the rotational orientation between the two prosthetic components that are connected to the present invention about a rotational axis. This is accomplished by providing a first member having an end comprising a channel. A second member is also provided having a crown for being laterally adjustably connected to the first member along an alignment axis and also rotationally adjustably connected to the first member along a rotational axis. In an alternative embodiment, scales are provided for measuring the adjustments made to the device.

This United States utility patent application claims priority on and the benefit of provisional application 61/766,339 filed Feb. 19, 2013, provisional application 61/710,441 filed Oct. 5, 2012, and provisional application 61/698,389 filed Sep. 7, 2012, the entire contents of each are hereby incorporated herein by reference.

### BACKGROUND OF THE INVENTION

1. Field of the Invention

The present invention relates to a device for offsetting two prosthetic components that are connected to opposite ends of the present invention wherein the first prosthetic component is laterally, rotatably (two independent adjustments), angularly and height adjustable in from the second prosthetic component a selected distance along an offsetting axis.

2. Description of the Related Art

Sometimes, due to accidents, health problems, birth defects, etc., people 5 need to have a limb 6 amputated. The amputated limb 6 terminates in a stump 7. In general, a socket 10 can be formed for any particular stump 7. Those sockets 10 are well known in the art, and each socket 10 has a central axis 11.

Fortunately for people requiring a prosthetic limb, much advancement has been made in the field of prosthetic limbs. Patients now have many choices, including endoskeletal and exoskeletal prosthetic limbs. The present invention relates generally to endoskeletal prosthetic limbs. That is, limbs comprised of structural components and that may have an optional aesthetic outer shell.

In the case of a prosthetic leg and foot combination, it is desirable that the prosthetic components of the leg be in predetermined angular alignment with respect to the prosthetic components of the foot.

The lower components can be out of alignment with the upper prosthetic components or the residual limb for other reasons as well. For example, the particular geometry of a person's limb 6 and stump 7 may cause the prosthetic components to be in an improper offset alignment, even if the prosthetics are connectable to the stump in a proper angular alignment.

Adjustments in the rotational alignment may also need to be achieved.

While advancements have been made, there is still room for advancement. Thus, there exists a need for an offset alignment device that solves these and other problems.

### SUMMARY OF THE INVENTION

The present invention relates to an offset alignment device with a longitudinal axis that is capable of singularly offsetting two prosthetic components a selected distance along an alignment axis that is selectably oriented in a plane that is generally perpendicular to the longitudinal axis, and is also capable of adjusting the rotational orientation between the two prosthetic components that are connected to the present invention about a rotational axis.

This is accomplished by providing a first member having an end comprising a channel. A second member is also provided having a crown for being laterally adjustably connected to the first member along an alignment axis and also rotationally adjustably connected to the first member along a rotational axis.

In this regard the angular alignment device is used to make adjustments in the lateral direction 15, the medial direction 16, the anterior direction 17 and/or the posterior direction 18. Diagonal and combinations of these directions can also be accomplished.

According to an advantage of the present invention, the lateral and rotational adjustments are independent of each other.

According to another advantage of the present invention, the crown is held in place within the channel as the third piece holds the crown tight within the channel.

According to a still further advantage of the present invention, the dome and pyramid are used to angularly offset the present invention with an adjacent component allowing for a third degree of adjustment.

According to a further advantage of the present invention, two independent rotational adjustments can be made.

According to a further advantage of the present invention, a lateral over-travel prevention mechanism is provided so that the part cannot inadvertently disassemble.

In another embodiment, the dome has ears that are received within the channel to provide additional stability to the device.

In another embodiment, scales are provided wherein the practitioner or user can measure exact adjustments within the devices.

In a further embodiment, the dome can be fixed to a slide brace with set screws. The slide brace can be movably received within a slide base and fixed with a screw that engages slots in the slide base to securely hold the parts together.

According to a further advantage of the present invention, a post can be secured through the slide base to be received within a limiter channel in the slide bracket to prevent the slide bracket from disengaging the slide base.

Other advantages, benefits, and features of the present invention will become apparent to those skilled in the art upon reading the detailed description of the preferred embodiments of the present invention and studying the drawings.

### BRIEF DESCRIPTION OF THE DRAWINGS

FIG. 1 is an exploded perspective view showing a configuration of the present invention in relation to existing prosthetic components.

FIG. 2 is a side exploded view of the present invention and two existing components.

FIG. 3 is a partial cross-sectional view of a four hole clamp on the end of a pylon.

FIG. 4 is a partial cross-sectional view of a threaded clamp on the end of three prong adapter.

FIG. 5 is a cross-sectional view of an embodiment of the present invention.

FIG. 6 is a top view of the present invention.

FIG. 7 is a bottom view of the present invention.

FIG. 8 is an exploded view of the present invention showing an angular offset, a lateral offset and a rotational offset.

FIG. 9 is a side view of the first member of the present invention.

FIG. 10 is a second side view of the first member of the present invention 90 degrees offset from the view of FIG. 9.

FIG. 11 is s bottom view of the first member of the present invention.

FIG. 12 is a cross-sectional view taken along line 12-12 in FIG. 10.

FIG. 13 is a side view of the second member of the present invention.

FIG. 14 is a cross-sectional view taken along line 14-14 in FIG. 13.

FIG. 15 is a top view of the second member of the present invention.

FIG. 16 is a bottom view of the second member of the present invention.

FIG. 17 is a side view of the third member of the present invention.

FIG. 18 is a cross-sectional view taken along line 18-18 in FIG. 17.

FIG. 19 is a top view of the third member of the present invention.

FIG. 20 is an exploded view of an alternative preferred embodiment of the present invention.

FIG. 21 is an alternative exploded view of the embodiment illustrated in FIG. 20.

FIG. 22 is a top view of the embodiment illustrated in FIG. 20.

FIG. 23 is a side view of the embodiment illustrated in FIG. 20.

FIG. 24 is an alternative side view of the embodiment illustrated in FIG. 20.

FIG. 25 is a perspective view of the embodiment illustrated in FIG. 20.

FIG. 26 is an alternative perspective view of the embodiment illustrated in FIG. 20.

FIG. 27 is an alternative perspective view of the embodiment illustrated in FIG. 20.

FIG. 28 is an alternative perspective view of the embodiment illustrated in FIG. 20.

FIG. 29 is a further alternative embodiment of the present invention.

FIG. 30 is an end view of the embodiment illustrated in FIG. 29.

FIG. 31 is a bottom view of the embodiment illustrated in FIG. 29.

FIG. 32 is a cross-sectional view taken along line 32-32 in FIG. 30.

FIG. 33 is a cross-sectional view taken along line 33-33 in FIG. 31.

FIG. 34 is a side view of the embodiment illustrated in FIG. 29.

FIG. 35 is a top view of the embodiment illustrated in FIG. 29.

FIG. 36 is a perspective view of a preferred slide base of the present invention.

FIG. 37 is a perspective view of the embodiment shown in FIG. 36.

FIG. 38 is a cross-sectional view taken along line 38-38 in FIG. 36.

FIG. 39 is a perspective view of a preferred slide bracket of the present invention.

FIG. 40 is a perspective view of the embodiment shown in FIG. 39.

FIG. 41 is a side view of the embodiment shown in FIG. 39.

FIG. 42 is a perspective view of a preferred pyramid post of the present invention.

FIG. 43 is a perspective view of the embodiment shown in FIG. 42.

FIG. 44 is a cross-sectional view taken along line 44-44 in FIG. 42.

FIG. 45 is a perspective view of a preferred slide bracket of the present invention.

FIG. 46 is a perspective view of the embodiment shown in FIG. 45.

FIG. 47 is a side view of the embodiment shown in FIG. 45.

### DETAILED DESCRIPTION OF THE PREFERRED EMBODIMENTS

While the invention will be described in connection with one or more preferred embodiments, it will be understood that it is not intended to limit the invention to those embodiments. On the contrary, it is intended to cover all alternatives, modifications and equivalents as may be included within the spirit and scope of the invention as defined by the appended claims.

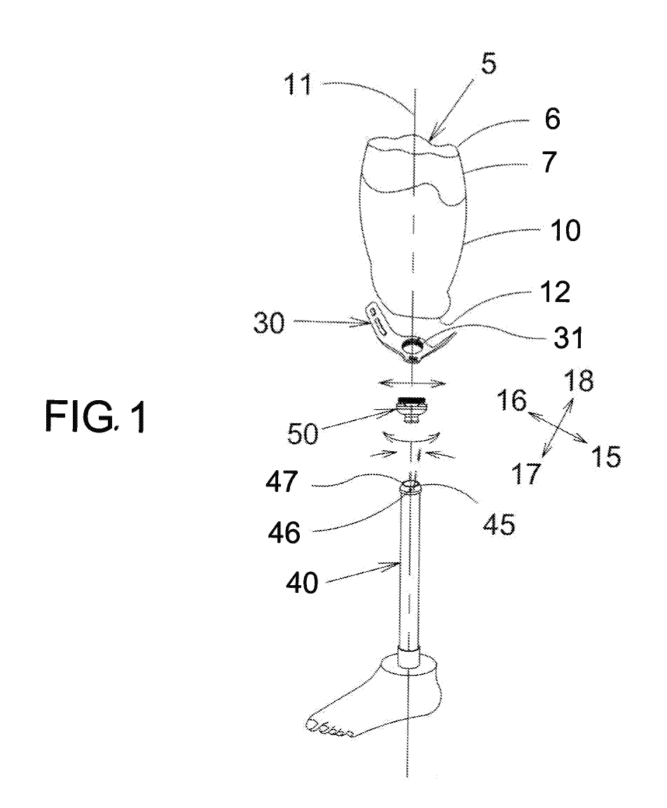

The present invention is intended for use with a prosthetic limb. A person 5 having a limb 6 terminating in a stump 7 may have prosthetic limb. FIG. 1 shows that a prosthetic limb has a socket 10 with a socket central axis 11 and an end 12. The socket 10 can be angularly offset from the remainder of the prosthetic component by angle alpha. A three prong adapter 30 having prongs, an internally threaded end clamp 31 and a central axis can be connected to the socket 10.

A pylon 40 with a fixed receiver 45 can be further provided. The fixed receiver 45 can be a four hole clamp having holes 46 through a sidewall and a rim 47 at the end of the clamp. The pylon can have a central axis. The central axis can be angularly offset from the three prong adapter central axis by angle beta. A person can adjustably select angle beta such that it is equal and opposite of angle alpha. In this regard, central axis of the pylon with a fixed receiver can be made parallel to, albeit offset from, the central axis 11 of the socket. This offset amount delta, can be in the lateral direction 15, the medial direction 16, the anterior direction 17 and the posterior direction 18. It offset amount can also be diagonal by being a combination of two of these directions.

The three prong adapter 30 may also be rotationally misaligned with a foot at the end of the pylon 40.

It is appreciated that while several components are described herein, that many alternative devices may also be used without departing from the broad aspects of the present invention.

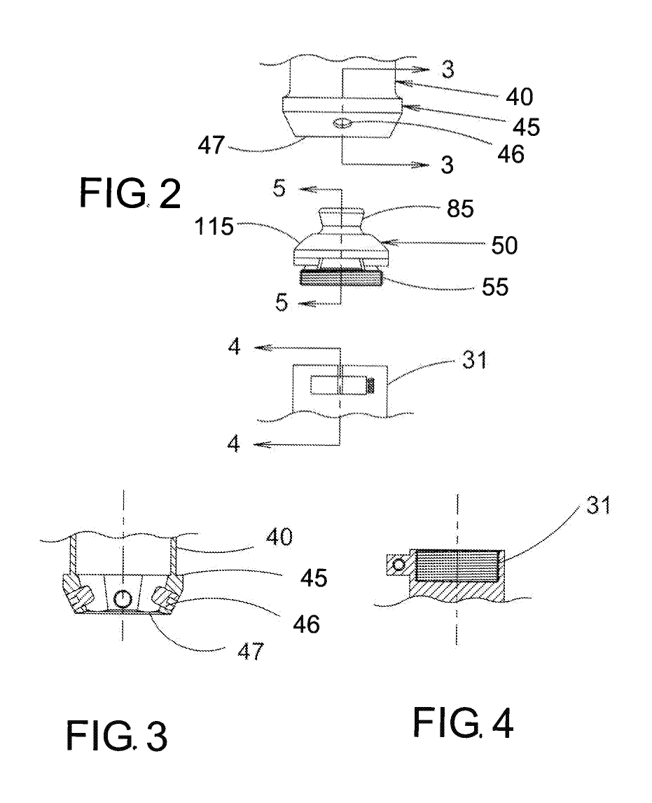

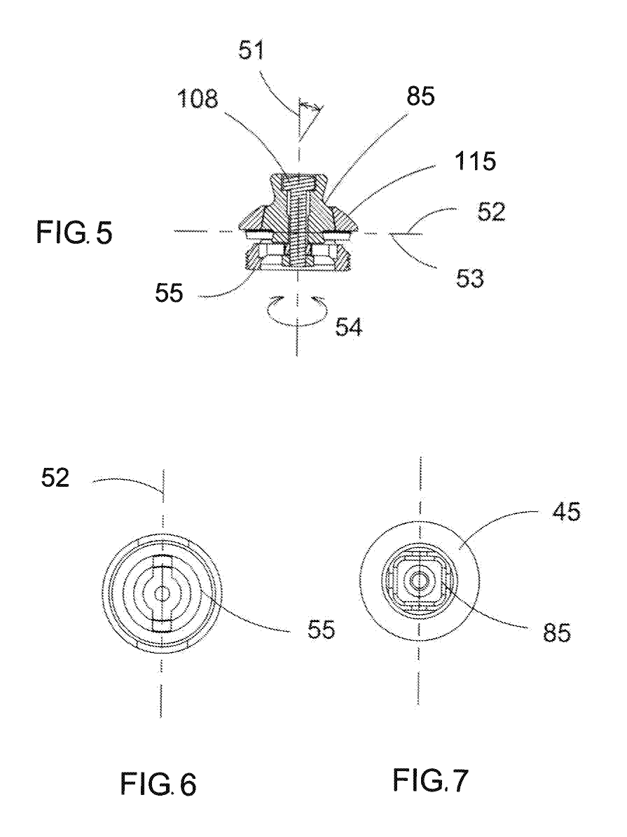

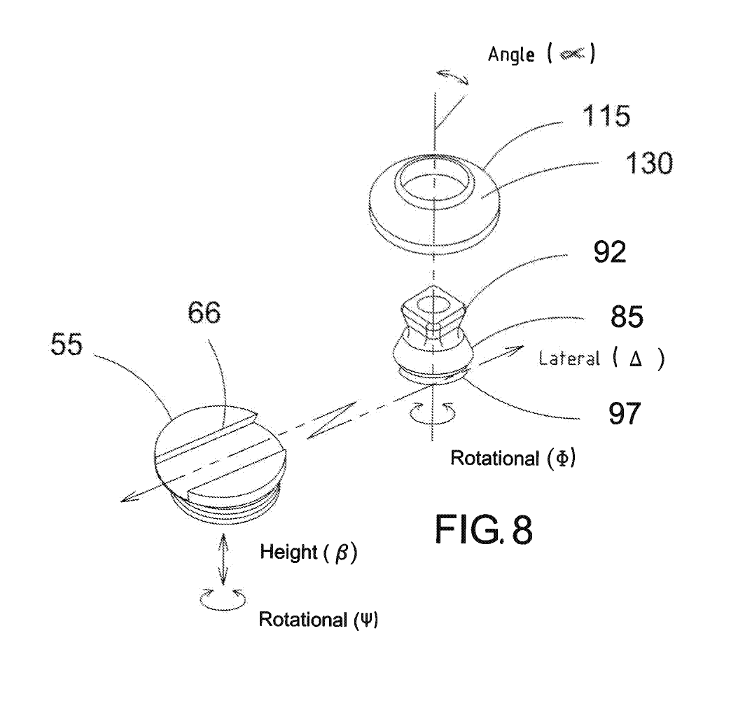

Turning now to the present invention, an offset alignment device 50 is provided. As shown in FIGS. 1, 2 and 5-8, the offset alignment device 50 generally has a first member 55, a second member 85 and a third member 115. The offset alignment device 50 further has a longitudinal axis 51 and an offsetting axis (lateral axis) 52 that lies in a plane 53. Plane 53 is preferably generally perpendicular to longitudinal axis 51. A rotational axis 54 is also provided. The rotational axis can be collinear with the longitudinal axis 51. The offset alignment device 50 is preferably made of steel or titanium allow. However, it can be made of other materials without departing from the broad aspects of the present invention. Further, the offset alignment device 50 is preferably made in a computer numeric controlled, or CNC, process. However, the offset alignment device 50 can be made by different processes without departing from the broad aspects of the present invention.

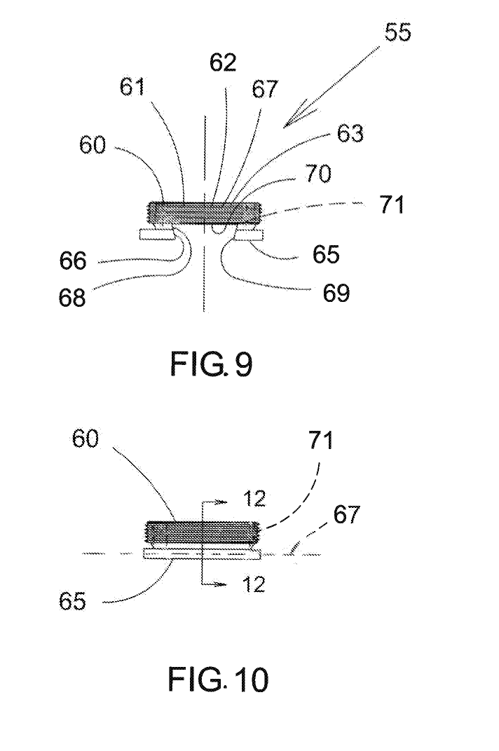

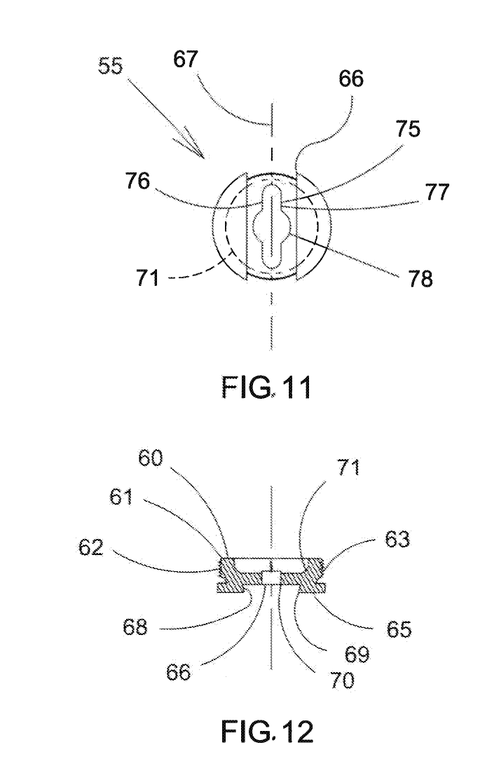

Looking now at the offset alignment device 50 of the present invention in more detail, the first member 55 is provided. As best shown in FIGS. 9-12, the first member 55 has a first end 60. The first end 60 of the first member 55 has a connector 61. In a preferred embodiment, the connector 61 has an exterior surface 62 that is threaded with threads 63. The first member 55 also has a second end 65. The second end 65 of the first member 55 has a channel 66 there through. The channel 66 has a channel longitudinal axis 67 that is preferably generally parallel to the offset alignment device 50 offsetting axis 52, and preferably generally perpendicular to the offset alignment device 50 longitudinal axis 51. The channel 66 has a first side 68, a second side 69 and a base 70. The first side 68 and second side 69 preferably extend from the base 70 at inwardly converging angles. However, the first side 68 and second side 69 could alternatively be generally perpendicular to the base 70. It is preferred that sidewalls 68 and 89 are smooth and without holes there through.

An internal sidewall 71 is also provided in the first end 60 of the first member 55 defining an internal void. A slot 75 is provided interior of the sidewall 71. The slot 71 preferably has generally parallel sides 76 and 77. A round central portion 78 is also provided. The slot spans in a direction generally parallel to the channel longitudinal axis 67.

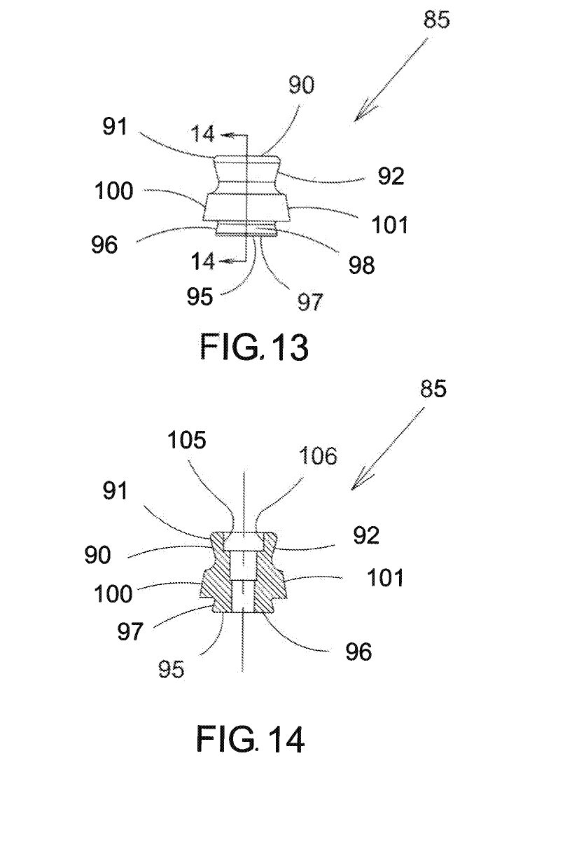

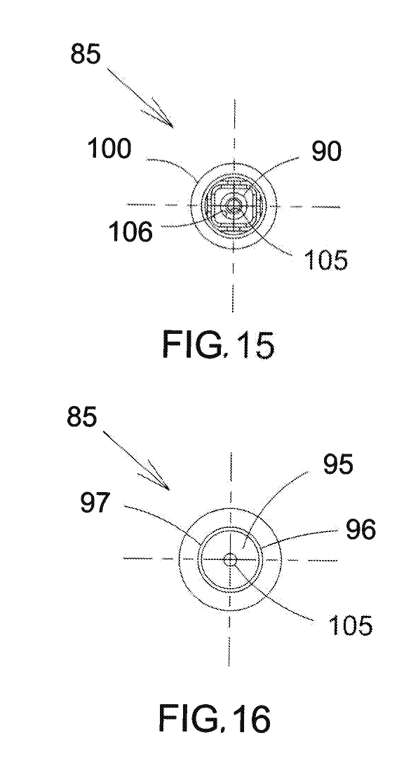

A second member 85 is also provided as is best shown in FIGS. 13-16. The second member 85 has a first end 90. The first end 90 of the second member 85 has a connector 91. In a preferred embodiment, the connector 91 comprises a pyramid 92. The second member 85 also has a second end 95. The second end 95 of the second member 85 comprises a connector 96 that is a crown 97. Crown 97 has a sidewall 98 that defines a generally circular shapes profile having a diameter slightly smaller than the width of the first member channel 66 between the sides 68 and 69. The sidewall 98 preferably diverges from the second end at an angle, such that the diameter of the crown is larger at the furthest remote portion of the crown.

A body 100 separates the first end 90 and second end 95. The body 100 has a sidewall 101 that is tapered in a conical dimension. The body sidewall 101 is wider adjacent the crown 97, and narrower adjacent the pyramid 92. A through hole 105 with a head area 106 is further provided along the longitudinal axis. The hole 105 receives a screw or bolt. The piece (screw, bolt or otherwise) is an over-travel prevention device. In this regard, the over-travel prevention device 108 prevents the crown 97 from being able to laterally slide out of the channel 66, as the device 108 would contact either end of the slot 75 before the crown escapes either end of the channel.

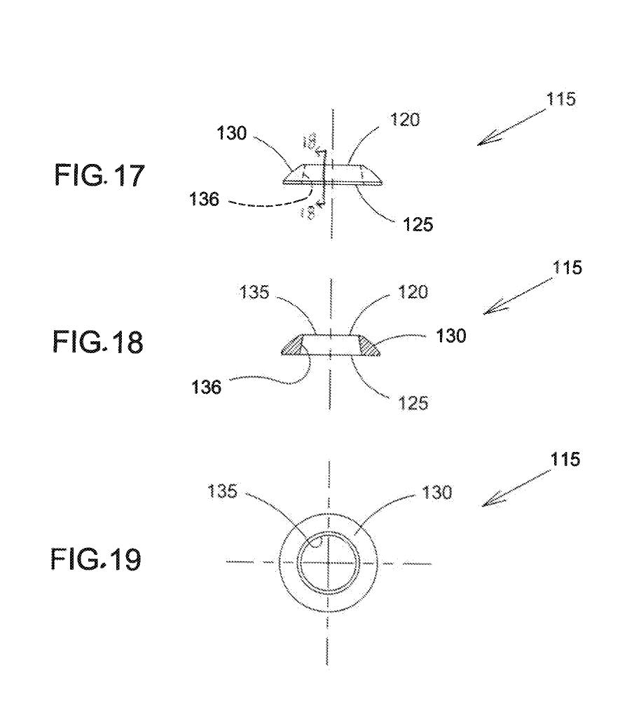

A third member 115 is also provided, as seen in FIGS. 17-19. The third member 115 has ends 120 and 125. A dome 130 is provided having a sloping sidewall. A hole 135 is preferably centrally aligned longitudinally through the third member. The hole 135 has a sidewall 136. The sidewall 136 is designed to mate with the body sidewall 101 of the second member 85.

The first member 55 is connectable to first prosthetic component. In a preferred embodiment, where the first member has a connector 61 comprising a threaded exterior surface 62, the first prosthetic component preferably has a complimentary internally threaded end. One example is a conventional three prong adapter 30 with internally threaded end 31. However, other prosthetic components with internally threaded ends could be used instead of the three prong adapter 30 without departing from the broad aspects of the present invention. In this regard, the first end 60 is threadably and rotatably connected to the first prosthetic component. Hence, the channel longitudinal axis 67 is adjustable to any selected orientation with respect to the first prosthetic component. The channel longitudinal axis 67 will return to the same selected orientation with respect to the first prosthetic component after each successive ½ revolution of the first member 55 with respect to the first prosthetic component. However, the channel will move closer to or away from the first prosthetic component depending on whether the first member 55 is threaded further onto the threaded end of the first prosthetic component. This allows for the height of the present invention to be fine-tuned.

The first member 55 is designed to laterally and adjustably connect to the second member 85. In a preferred embodiment, this is accomplished by sliding the crown 97 of the second member 85 within the channel 66 of the first member 55. The crown 97, by virtue of being round, is able to be rotationally adjusted independent of the lateral positioning of the crown, wherein any desired rotational orientation can be achieved. It is appreciated that the rotation of crown 97 within the channel 66 is independent of the rotation of the first member relative the adjacent prosthetic component. In this regard, the practitioner has two independent options available for making rotational adjustments.

The second member 85 is connectable to a second prosthetic component to lock the first member 55 in desired lateral and rotational position (phi) relative the second member 85, and also to lock the alignment device 55 in a selected angular alignment with the adjacent component. In a preferred embodiment, where the second member 85 has a connector 91 comprising a pyramid 92, the second prosthetic component preferably has a pyramidal receiver. One suitable example is a conventional pylon 40 with fixed pyramidal receiver 45. However, other prosthetic components with pyramidal receivers can be used without departing from the broad aspects of the present invention. The pyramid 92 of the second member 85 is angularly adjustable relative to the pyramidal receiver.

The pyramidal receiver or clamp has a rim 47 that contacts the dome 130 of the third member 115. In this regard, the dome 130 is held in place by the rim 47 of the adjacent component any the selected angular alignment, as the force of the set screws forces the adjacent component into secure contact with the dome 130. The set screws also force the pyramid upward as the force the dome downward. This frictionally locks the second member 85 in its lateral and rotational orientation relative the first member 55. It is appreciated that this locking occurs without any set screws passing into the channel to contact the crown.

The alignment device 50 aligns two prosthetic components that are connected to it. In this regard, the offset alignment device 50 is capable of adjusting a distance delta to overcome any offsetting distance between two prosthetic components. This is accomplished by making an adjustment along a single offsetting axis 52. The present invention is also useful in overcoming angular and rotational offset conditions.

In summary, the present invention allows for at least 5 adjustments: namely, 1) rotation (psi) between the first member and the adjacent component; 2) height (beta) of the overall component because of the threadable connection between the alignment device and the adjacent component; 3) lateral travel (delta) (limited by a limiting device 108) between the first and second members; 4) rotational orientation (phi) between the first and second members; and 5) angular adjustment (alpha) between the second member and its adjacent prosthetic component.

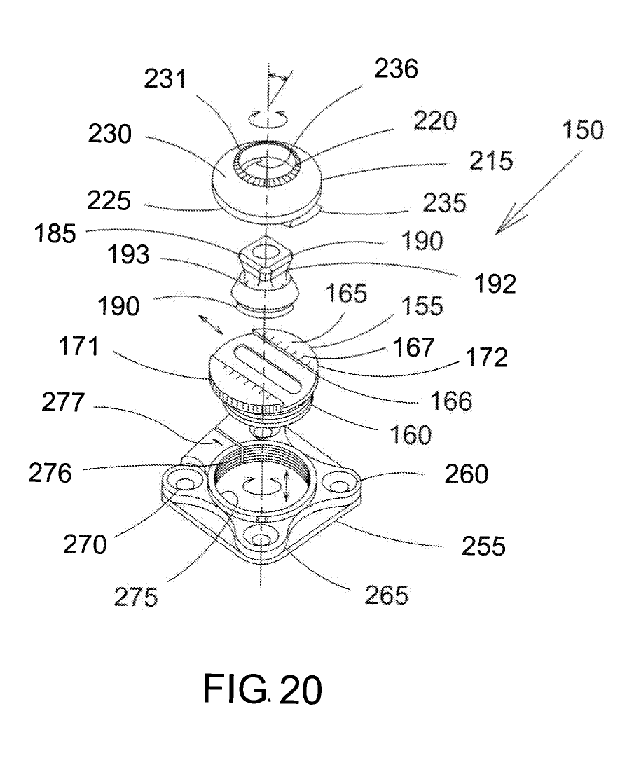

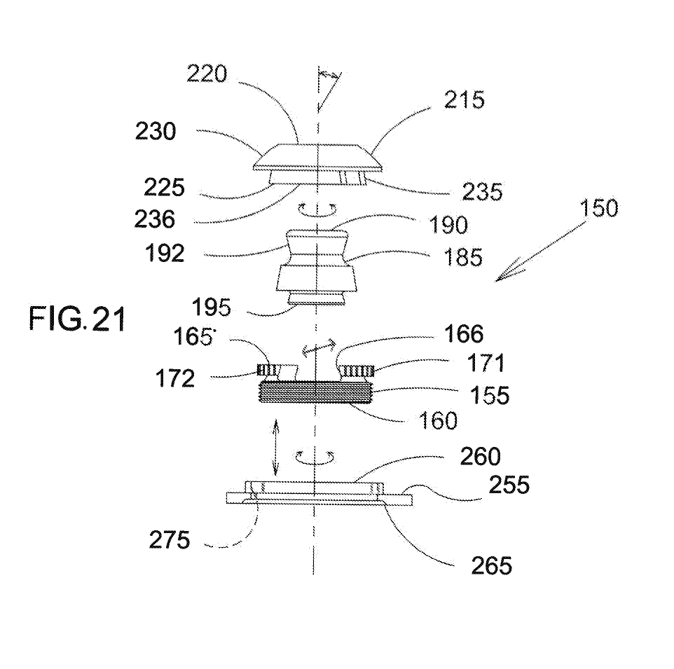

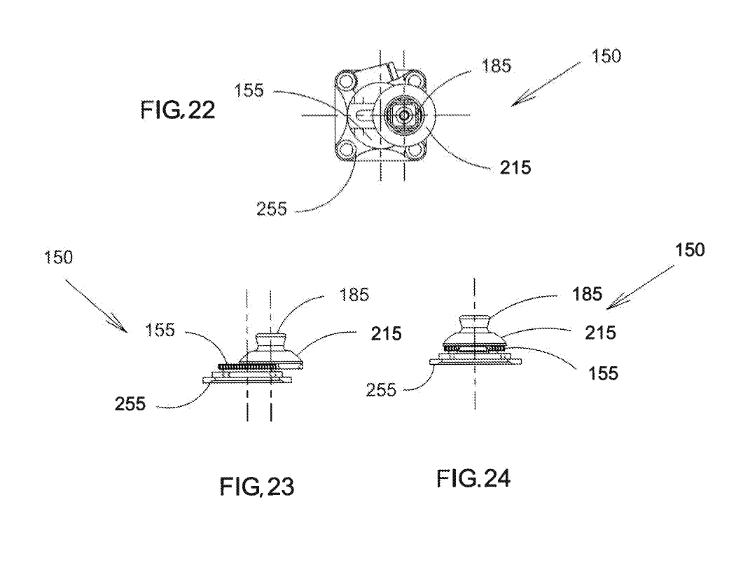

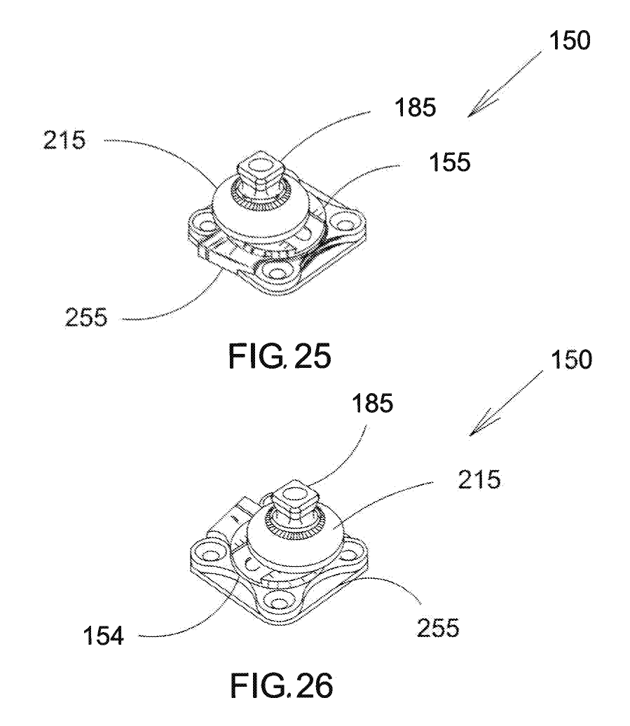

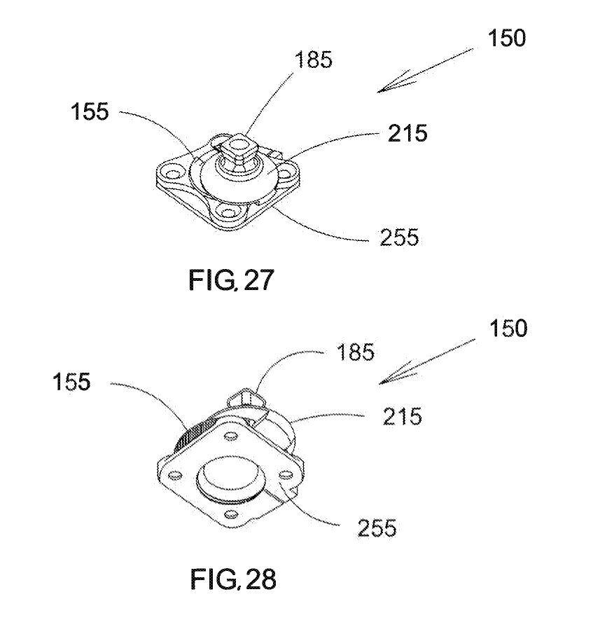

Turning now to FIGS. 20-28, it is seen that an alternative preferred embodiment of the present invention is illustrated.

An alignment device 150 having four members 155, 185, 215 and 255 is provided. Members 155, 185 and 215 are similar to the members of the embodiment 50 described above. Some distinctions are described below.

Member 155 has ends 160 and 165. A channel 166 is provided. A scale 167 is provided on the side of the channel. Scale 167 can be a series of markings formed into the members at predetermined spacing. In the preferred embodiment, the spacing between markings can be 5 mm. Yet, it is understood that the spacing can be larger or smaller without departing from the broad aspects of the present invention. A sidewall 171 with a scale 172 is also provided. Scale 167 is a linear scale. Scale 172 is a rotational scale. Scale 172 preferably has markings every 10 degrees. Yet, the markings can be more or less frequent without departing from the broad aspects of the present invention.

Member 185 has a first end 190 and a second end 195. A pyramid 192 is provided having an etching (or marking) 193 at the base of the pyramid. It is understood that a protrusion or other indicator could be provided without departing from the broad aspects of the present invention.

Member 215 has ends 220 and 225. A dome 230 is provided as is a scale 231. The scale 231 is at the top of the dome 230 and has markings circumferentially space at 10 degree intervals around a central hole. Yet, the markings can be more or less frequent without departing from the broad aspects of the present invention. Ears 235 and 236 are at the second end of the member 215. The ears are shaped to be received within the channel 166. The ears prevent the member 215 from rotating relative member 155. In this regard, the measure from the scale 231 is accurate and easily determinable relative the other members.

The etching 193 of the second member can be aligned with the markings of scale 231 so that the rotational orientation of the members 185 and 215 can be determined.

The second and third members 185 and 215, respectively, can be longitudinally moved within the channel a selected linear distance. The selected linear distance can be measured by the scale 172 adjacent the channel 171.

The fourth member 255 has ends 260 and 265. There are preferably four connecting holes 170 located in the corners of the generally square profile shaped member. A threaded central hole 275 is further provided. A clamp 276 and an etching 277 are further provided. The clamp 276 can be used to constrict the size of the opening 275 to tighten onto an adjacent component, preferably the first member 155. The etching 277 indicates the amount of angular rotation is present (via comparison with scale 172) between the first and fourth members. Also, the height between the first and fourth members is adjustable by making successive rotations of the first member threaded end within the threaded clamp of the fourth member.

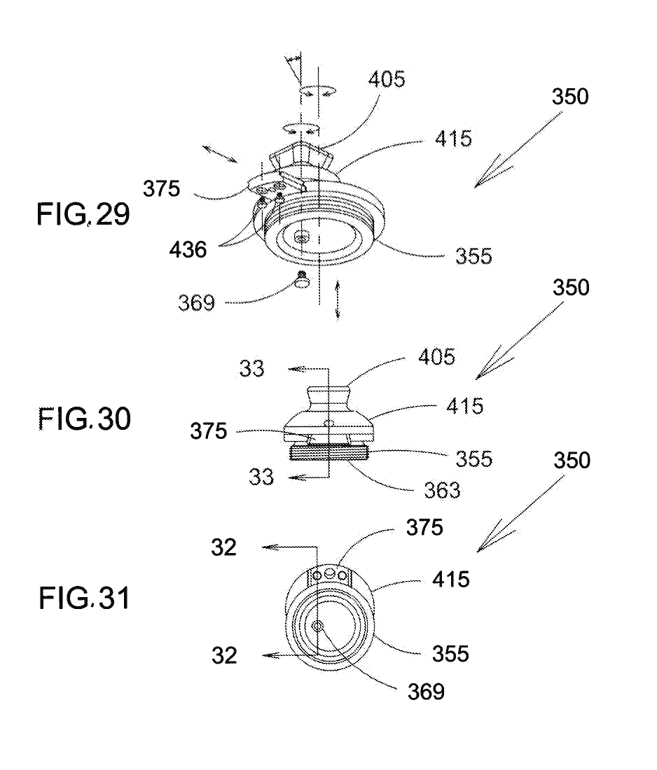

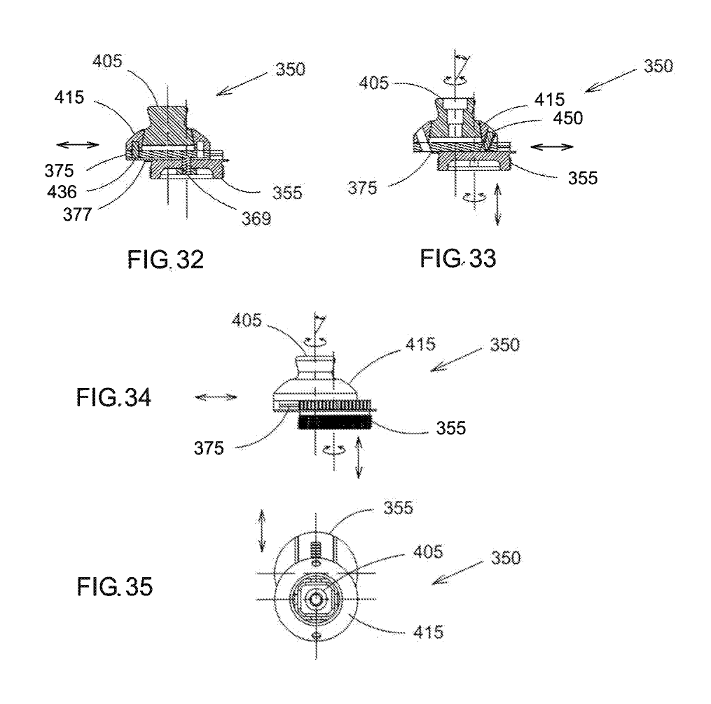

Looking now at FIGS. 29-47, it is seen that an additional preferred embodiment is illustrated. In this embodiment, an alignment device 350 having a slide base 355, a slide bracket 375, a pyramid post 405 and a dome member 415 is provided. These parts are shown in a connected configuration in FIGS. 29-35. It is understood that while one position is shown in these figures, that many adjustments can be made as provided by the present invention.

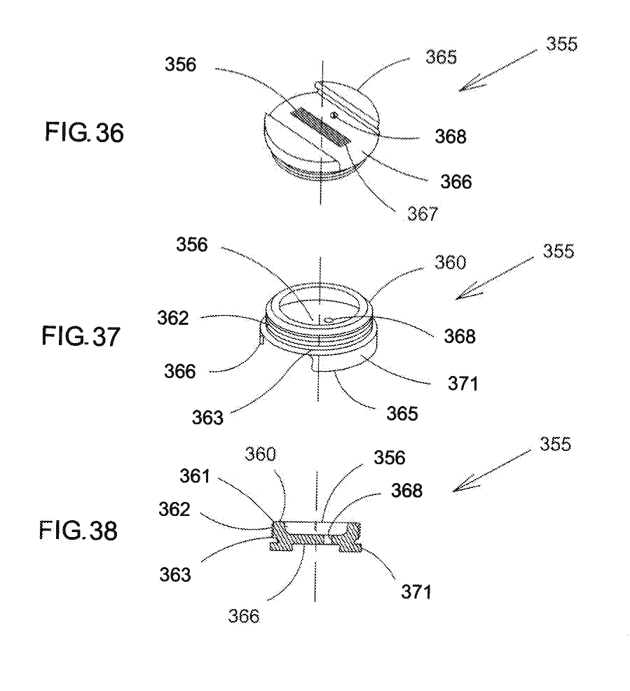

Looking now specifically at FIGS. 36-38, it is seen that a preferred first member of the alignment device 350 is a slide base or slide base member 355. Slide base 355 has a first end 360 with a connector 361 thereon. Connector 361 preferably has an exterior surface 362 with threads 363 thereon. The first end 360 can be connected to an adjacent prosthetic component such as a component having a clamp that can securely attach to the first end 360 when the member is rotated to the selected rotational orientation. It is appreciated that such a connection can control height via advancement on the threads as well as one form of independent rotation. In this regard, the independent rotation rotates all the parts connected to the slide base in a slide base orbit about the slide base center point 356.

The slide base 355 also has a second end 365. The second end 365 has a channel 366 formed therein with a longitudinal axis. The channel 366 preferably has sidewalls with recessed portions. The channel further has a bottom with slots 367. There is a plurality of slots that extend preferably the majority of the length of the channel 366. The slots 367 are preferably perpendicular to the channel longitudinal axis. A hole 368 (perpendicular to both the longitudinal axis and the slots 367) is further provided. The hole extends from the second end 365 into the channel 366 and can receive a screw 369 or bolt. The second end further has a sidewall 371. The channel extends through the sidewall 371 on both ends of the channel 366.

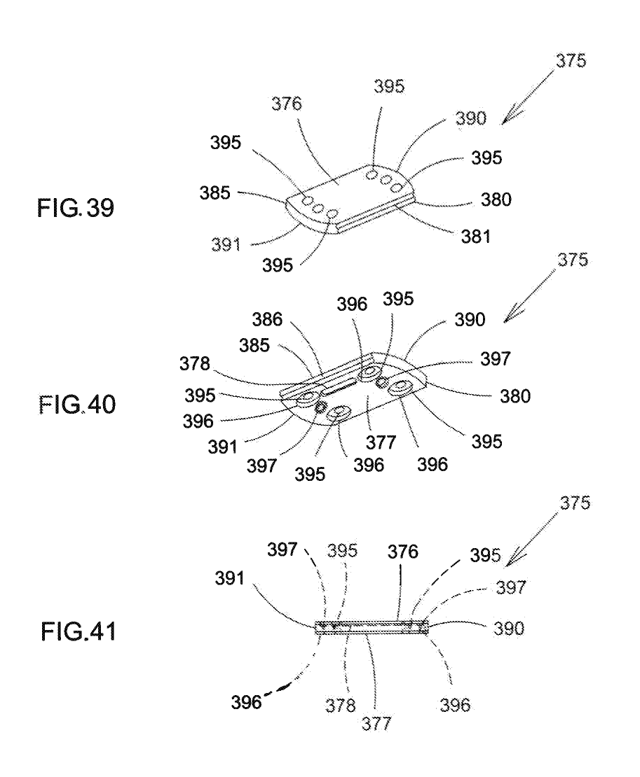

Turning now to FIGS. 39-41, it is seen that a second member is provided. The second member is a slide bracket or slide bracket member 375. The slide bracket 375 preferably has a top 376 and a bottom 377. A channel 378 is preferably located on the bottom 377 of the slide bracket 375. The channel 378 is parallel to a first side 380 and a second side 385. The channel 378 is preferably closer to the first side 380. The first side 380 has a side profile 381 and the second side 385 has a second side profile 386. The profiles, 385 and 386, respectively, are shaped to slidingly mate with the walls of channel 366 whereby the slide bracket 375 is slidably adjustable relative the slide base 355. The slide bracket further has two ends 390 and 391 respectively. Four perpendicular holes 395 are preferably provided adjacent the corners of the slide bracket 375. Each hole 395 has a relieved area on the bottom 377 side of the bracket 375. Two angle holes 397 are further provided for receiving screws. The screws through angle holes 397 engage the slots 367 of the base 355 to secure the slide bracket 375 into a selected position relative the slide base 355 along the longitudinal axis of the channel 366.

Screw 369 passing through hole 368 extends into channel 378 when the slide bracket is within slide base channel 366. The screw 369 keeps the bracket from extending too far laterally relative the base, as the screw will engage then ends of the channel 378 to limit the travel.

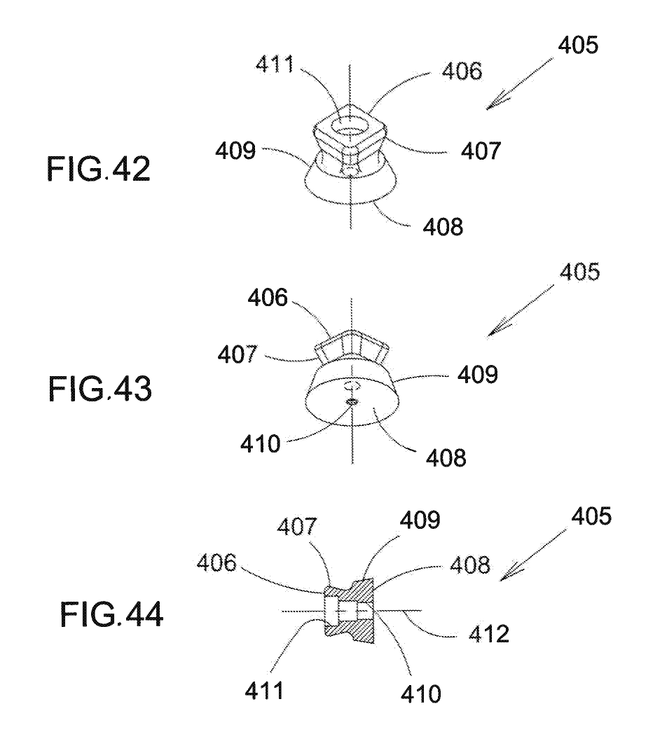

Turning now to FIGS. 42-44, it is seen that a third member is provided. The third member can be a pyramid post or pyramid post member 405. Pyramid post 405 has a first end 406 with a pyramid 407 thereon. The pyramid 407 preferably has four faces that are outwardly diverging so that it can be secured to an adjacent component in a secure matter. It is appreciated that while four faces are shown, the more or fewer faces, or even a smooth surface, could be provided without departing from the broad aspects of the present invention. The pyramid post 405 further has a second end 408. A side wall 409 spans between the second end 408 and the pyramid 407. The side wall is preferably cone shaped. A hole 410 is preferably centrally aligned through the pyramid post 405. A relieved or clearance area 411 is provided at the top of the pyramid 407 to allow the head of a screw or bolt to be recessed within the pyramid. The hole 410 is along the post central axis 412 and also passes out the second end. 408 of the pyramid post.

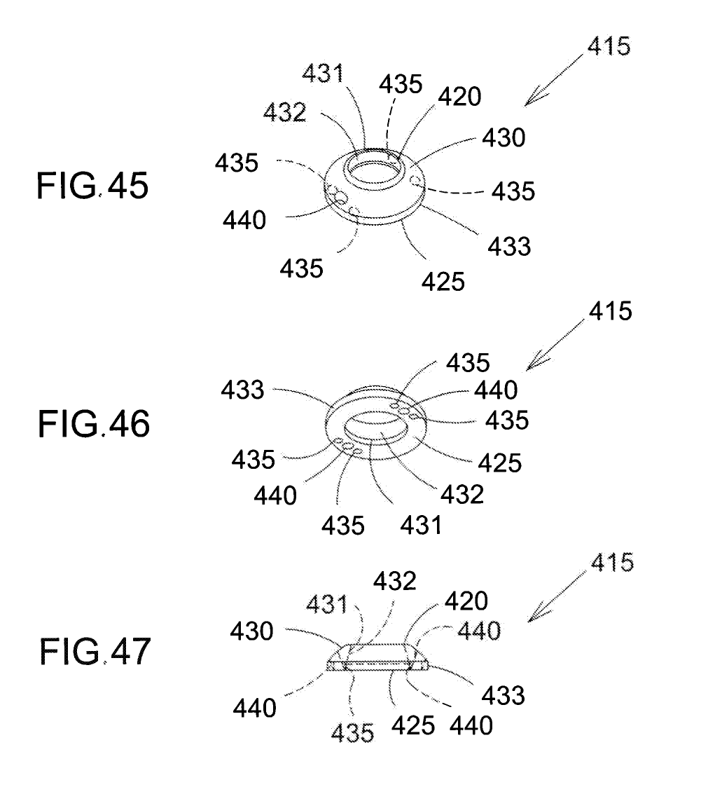

Turning now to FIGS. 45-47, it is seen that a fourth member is provided, which is preferably a dome member 415. Dome member 415 has a first end 420 and a second end 425. A dome 430 is on first end 420. A central hole 430 is provided with a side wall 432 that has a converging profile. In this regard the sidewall has a larger diameter at end 425 than it does at end 420. A flat side wall portion 433 is at the bottom of the dome 430 between the dome and the second end 425 of the dome member 415.

Four perpendicular holes 435 are formed into the second end 425 of the dome member 415. The perpendicular holes are perpendicular to the bottom surface and are spaces to align with the four perpendicular holes 395 of the slide bracket 375. In this regard, the dome member 415 is removably fixed to the slide bracket 375 via the respective holes as screws 436 can be inserted through the holes. It is understood that more or fewer holes, and that the angular alignment of the holes relative their respective parts may be varied without departing from the broad aspects of the present invention.

Two angled holes 440 are further provided. The angled holes preferably extend through the dome 430 out the second end 425 of the dome member 415. Angled holes 440 align with angle holes 397 of the slide bracket 375. In this regard, one or two screws 450 are provided to pass through the dome member 415 and the slide bracket 375 to engage the slots 367 of the slide base 355 to fix these components relative the channel in the desired offset location or position.

The pyramid post 405 is rotatably held within the dome member. The post 405 can rotate on an independent axis relative the dome member 415. The post is fixed with the dome member when the adjacent prosthetic component engages the pyramid and forces the sidewall 409 of the pyramid post into mating engagement with the side wall 432 of the central hole 431 of the dome member 435.

In this regard, the pyramid post 415 is able to be orbited about the central point 356 of the slide base 355 as the slide base rotates about the adjacent component. Second, the pyramid post 415 rotates about its own central axis 412 within the domed member 415. In this regard, present invention is capable of two independent forms of rotation, in addition to lateral adjustment, height adjustment and angular adjustment on the dome.

It is understood that numerical members (such as first member, second member, etc.) are provided for illustrative purposes, and as such can have different names without departing from the broad aspects of the present invention.

Thus it is apparent that there has been provided, in accordance with the invention, a device for offsetting prosthetic components that fully satisfies the objects, aims and advantages as set forth above. While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims.

1. A prosthetic component comprising:
a first member;
a second member;
a third member; and
a fourth member,
wherein:
said first member is rotatably connected to a prosthetic component;
said second member is laterally connected to said first member;
said third member is rotatable relative said fourth member; and
said fourth member is connected to said second member.

2. The prosthetic component of claim 1 wherein said first member is a slide base.

3. The prosthetic component of claim 2 wherein said slide base has a threaded exterior surface at one slide base end, and a channel on a second slide base end.

4. The prosthetic component of claim 3 wherein said channel comprises slots.

5. The prosthetic component of claim 1 wherein said second member is a slide bracket.

6. The prosthetic component of claim 5 wherein said slide bracket has a bracket top and a bracket bottom, wherein said bracket bottom has a second bracket channel therein.

7. The prosthetic component of claim 5 wherein said first piece comprises a screw for being received within said second bracket channel, wherein motion of said slide bracket relative said slide base is restricted.

8. The prosthetic component of claim 1 wherein said fourth member is a dome member.

9. The prosthetic component of claim 8 wherein said dome member comprises a plurality of perpendicular holes, wherein at least one of said plurality of perpendicular holes receives a screw to attach said dome member to said second component.

10. The prosthetic component of claim 9 wherein said dome member comprises at least one angle hole, said at least one angle hole receiving a screw to fix said dome in lateral relation to said first member.

11. (canceled)

12. A prosthetic component comprising:
a slide base;
a slide bracket laterally movable relative said slide base;
a pyramid post held relative said slide bracket; and
a dome member, said dome member being connected to said slide bracket.

13. The prosthetic component of claim 12 wherein said slide base has a threadable end, wherein said prosthetic component can be rotation about said threaded end.

14. The prosthetic component of claim 13 wherein said pyramid post is rotatable relative said dome member, whereby said prosthetic component has two independent axis of rotation.

15. The prosthetic component of claim 12 further providing at least one scale.

16. A prosthetic component comprising:
a base having a threadable connector providing a first axis of rotation; and
a post, said post being rotatable relative said base providing a second axis of rotation,
whereby said second axis of rotation is independent of said first axis of rotation.

17. The prosthetic component of claim 16 wherein said post is laterally adjustable relative said base.

18. The prosthetic component of claim 16 further comprising a dome, said dome housing said post.

19. The prosthetic component of claim 16 wherein said base comprises a channel.

20. The prosthetic component of claim 16 wherein said threadable connector also provides height adjustment to said prosthetic component.

21. The prosthetic component of claim 1 wherein at least one of said first member, said second member, said third member and said fourth member comprises a scale.
